(19) United States
(12) Patent Application Publication
(10) Pub. No.: US 2024/0225931 A9
(43) Pub. Date: Jul. 11, 2024

(54) APPARATUS COMPRISING AN INCLINE BED AND METHOD FOR TREATING SYMPTOMS OF PATIENTS WITH CHRONIC DISORDERS

(71) Applicant: Miguel Angel Bravo Gutierrez, Monterrey (MX)

(72) Inventor: Miguel Angel Bravo Gutierrez, Monterrey (MX)

(21) Appl. No.: 18/381,676

(22) Filed: Oct. 19, 2023

Related U.S. Application Data
(60) Provisional application No. 63/418,523, filed on Oct. 22, 2022.

(51) Int. Cl. A61G 7/018; A61G 7/005; G16H 40/63

(52) U.S. Cl. CPC A61G 7/018; A61G 7/005; G16H 40/63; A61G 2203/12

(57) ABSTRACT

The present invention relates to the field of systems for the treatment of patients with chronic disorders and aims to provide an apparatus that allows the complete inclination of a mattress, as well as the method for the non-invasive treatment of some diseases involving gastrointestinal disorders, respiratory and cardiovascular, through routines that involve the combination of two variables: time and patient angulation. In addition to the above and based on the patient's feedback on the progress and results of this therapy, as well as the monitoring of vital signs and the consolidation of the information shared by other users for Big-Data analysis, it will be possible to achieve predictive behaviors that help users improve their symptoms or chronic disorders or definitely cure themselves completely through tilt therapy as part of existing non-invasive and/or adjuvant methods.

### FIELD OF THE INVENTION

The present invention relates to the field of systems for the treatment of patients with chronic disorders, specifically it relates to a tilting bed for treating symptoms of chronic diseases and a method for adjusting the angle based on feedback from the patient and the vital signs monitoring.

### BACKGROUND OF THE INVENTION

Currently, there are various disorders and chronic diseases that require periodic attention when the patient is in a resting position, particularly lying in a bed in a flat position, because symptoms related to them may arise or worsen. These disorders and diseases we find can be classified into several groups, those of gastrointestinal origin, such as gastroesophageal reflux disease (GERD^{1,2,3,4,5,6,7,8,9}) those related to the respiratory system such as obstructive sleep apnea (OSA) and some others that can be related to the cardiovascular system or postural orthostatic tachycardia syndrome (POTS^{10,11,12,13,14}), among others. ¹Arin, A. and Iglesias, MR Gastroesophageal reflux disease. AnalesSis San Navarra [online]. 2003, vol. 26, n.2 [cited 2022 Nov. 14], pp. 251-268.²Eusebi L H, Black C J, Howden C W, Ford A C. Effectiveness of management strategies for uninvestigated dyspepsia: systematic review and network meta-analysis. BMJ. 2019; 367:16483.³CalvetaX, PonceJ. Gastroesophageal reflux disease: epidemiology, diagnosis and treatment. Elsevier, 2006, Vol. 29, No. S3, pp. 23-30.⁴Albarqouni L, Moynihan R, Clark J, Scott A M, Duggan A, Del Mar C. Head of bed elevation to relieve gastroesophageal reflux symptoms: a systematic review. BMC Fam Pract. 2021 Jan. 19; 22(1):24. doi:10.1186/s12875-021-01369-0. PMID: 33468060; PMCID: PMC7816499.⁵Huerta-Iga, Tamayo de la Cuesta, et al. Mexican Consensus on gastroesophageal reflux disease. RevistadeGastroenterologiadeMixico.2012; 77(4):193-213.⁶Kaltenbach T, Crockett S, Gerson L B. Are lifestyle measures effective in patients with gastroesophageal reflux disease? An evidence-based approach. Arch Intern Med. 2006; 166(9):965-971.⁷Person E, Rife C, Freeman J, ClarkA, Castell D O. A novel sleep positioning device reduces Gastroesophageal reflux: a randomized controlled trial. J Clin Gastroenterol. 2015; 49(8):655-659.⁸Singh M, Lee J, Gupta N, Gaddam S, Smith B K, Wani S B, et al. Weight loss can lead to resolution of gastroesophageal reflux disease symptoms: a prospective intervention trial. Obesity (Silver Spring) 2013; 21(2):284-290.⁹Piesman M, Hwang I, Maydonovitch C, Wong R K. Nocturnal reflux episodes following the administration of a standardized meal. Does timing matter?Am J Gastroenterol. 2007; 102(10):2128-2134.¹⁰Harris C I. COVID-19 Increases the Prevalence of Postural Orthostatic Tachycardia Syndrome: What Nutrition and Dietetics Practitioners Need to Know. J Acad Nutr Diet. 2022 September; 122(9):1600-1605. doi: 10.1016/j.jand.2022.06.002. Epub 2022 Jun. 10. PMID: 35697326; PMCID: PMC9186518.¹¹Eldokla A M, Mohamed-Hussein A A, Fouad A M, Abdelnaser M G, Ali S T, Makhlouf N A, Sayed I G, Makhlouf H A, Shah J, Aiash H. Prevalence and patterns of symptoms of dysautonomia in patients with long-COVID syndrome: A cross-sectional sectional study. Ann Clin Transl Neurol. 2022 June; 9(6):778-785. doi:10.1002/acn3.51557. Epub 2022 Apr. 8. PMID: 35393771; PMCID: PMC9110879.¹²Dysautonomias: Clinical Disorders of the Autonomic Nervous System. Moderator: David S. Goldstein, MD, PhD; Discussants: David Robertson, MD; Murray Esler, MD; Stephen E. Straus, MD; and Graeme Eisenhofer, PhD.¹³Abed H, Ball P A, Wang L X. Diagnosis and management of postural orthostatic tachycardia syndrome: A brief review. J Geriatr Cardiol. 2012 March; 9(1):61-7. doi: 10.3724/SP.J.1263.2012.00061. PMID: 22783324; PMCID: PMC3390096.¹⁴Fu Q, VanGundy T B, Galbreath M M, et al. Cardiac origins of the postural orthostatic tachycardia syndrome. JAm Coll Cardiol. 2010; 55:2858-2868.

Obstructive sleep apnea is a disorder that affects 17 to 24% of American adults¹⁵. It is conservatively estimated that OSA is prevalent at 3% among women and 10% among men aged 30 to 49 years; while 9% among women and 17% among men ages 50 to 70, including about 24 million people in the United States who have not received a diagnosis¹⁶. The prevalence of OSA has increased substantially from original reports of 2% in women and 4% in men to 23 to 26% in women and 40.6 to 49.7% in men¹⁷ confirmed by polysomnography. In other words, apnea affects almost one in four men and one in ten women, between 30 and 60 years old. The health consequences caused by this disorder include neuropsychiatric and cardiovascular sequelae that alter professional, family and social life and negatively impact health-related quality of life¹⁸, in addition to being associated with neurohormonal and electrophysiological abnormalities that can increase the risk of sudden cardiac death, especially during sleep¹⁹. ¹⁵ Young T, Peppard P E, Gottlieb D J. Epidemiology of obstructive sleep apnea: a population health perspective. Am J Respir Crit Care Med 2002; 165:1217-39.¹⁶ Young T, Palta M, Dempsey J, Skatrud J, Weber S, Badr S. The occurrence of sleep-disordered breathing among middle-aged adults. N Engl J Med. 1993; 328(17):1230-5. https://doi.org/10.1056/NEJM199304293281704.¹⁷ Heinzer R, Marques-Vidal P, Marti-Soler H, Andries D, Tobback N, Mooser V, et al. Prevalence of sleep-disordered breathing in the general population: the HypnoLaus study. Lancet Respir Med. 2015; 3(4):310-8. https://doi.org/10.1016/S2213-2600(15)00043-0.¹⁸ Moyer C A, Sonnad S S, Garetz S L, Helman J I, Chervin R D. Quality of life in obstructive sleep apnea: a systematic review of the literature. Sleep Med. 2001 November; 2(6):477-91. doi: 10.1016/s1389-9457(01)00072-7. PMID: 14592263.¹⁹Day-Night Pattern of Sudden Death in Obstructive Sleep ApneaMar. 24, 2005 N Engl J Med 2005; 352:1206-1214 DOI: 10.1056/NEJMoa041832.

There are various alternatives to treat OSA, from the use of specialized technology such as positive pressure machines to correcting the position at bedtime, by placing devices in the bed so that the patient can remain in a certain position that helps prevent the tongue from obstructing the airways.

Studies have shown that there has been a 31.8% reduction in the apnea-hypopnea index (AHI) when there is mild elevation of the head of the bed. Mild elevation of the head of the bed has significantly reduced AHI from 15.7 to 10.7 events per hour of 7.5° significantly improves the severity of obstructive sleep apnea (OSA) without interfering with sleep architecture. Elevating the head of the bed has been shown to be a simple alternative treatment to improve OSA, as there is evidence that body position and sleep stage significantly influence the level of positive pressure needed to treat obstructive respiratory abnormalities²⁰. ²⁰Souza FJFB, Genta P R, de Souza Filho A J, Wellman A, Lorenzi-Filho G. The influence of head-of-bed elevation in patients with obstructive sleep apnea. Sleep Breath. 2017 December; 21(4):815-820. doi:10.1007/s11325-017-1524-3. Epub 2017 Jun. 24. PMID: 28647854; PMCID: PMC5700252.

Pressure level requirements may vary over time due to various factors, such as weight gain or loss, use of medications and alcohol, nasal congestion, changes in jaw position (for example, due to a MAD), during the alternating cyclic pattern of sleep stages, the position of the body during rest and within CPAP therapy (CPAP is believed to play a role in reducing edema resulting from the vibration associated with snoring and mouth sucking upper respiratory tract induced apnea)^{21,22,23,24}. ²¹Ravesloot M J L, de Vries N. Reliable calculation of the efficacy of non-surgical and surgical treatment of obstructive sleep apnea revisited. Sleep. 2011; 34:105-110.²² Pevernagie D J, Shepard J W. Relationships between sleep stage, posture and effective nasal CPAP levels in OSA. Sleep. 1992; 15:162-167.²³Sériès F, Marc I. Importance of sleep stage- and body position-dependence of sleep apnea in determining benefits to auto-CPAP therapy. Eur RespirJ. 2001; 18:170-175. doi: 10.1183/09031936.01.98103501.²⁴Oksenberg A, Silverberg D S, Arons E, Radwan H. The sleep supine position has a major effect on optimal nasal continuous positive airway pressure. Chest. 1999; 116:1000-1006. doi:10.1378/chest.116.4.1000.

Patients with reflux are more likely to have Barrett's esophagus, the younger the age at onset of symptoms the longer the duration of symptoms and the more severe the episodes of nocturnal reflux. The prevalence of Barrett's esophagus is higher in men and its incidence increases with age.

Among the common symptoms of this disorder are heartburn or pyrosis, dyspepsia and regurgitation are common: one in five adults reports one of these symptoms at some point in their life; and they are a very frequent reason for consultation in primary care. Guidelines for the treatment of GERD recommend a gradual or stepwise approach to managing your symptoms, starting with nonpharmacologic interventions that include lifestyle modifications (e.g., weight loss, smoking cessation, and avoiding late meals or at night) and progress to surgical and medical interventions when necessary. However, the widespread use of pharmacological interventions has made non-pharmacological interventions underutilized and obsolete, and their full potential as an effective complement or alternative to pharmacological interventions has been under-researched and underutilized.

A promising, easy-to-adopt, non-pharmacological intervention to treat GERD is elevating the head of the bed. Clinical trials have been conducted to evaluate this simple intervention; However, evidence from a recent high-quality systematic review summarizing evidence to inform practice on non-pharmacological measures is limited. There is a systematic review of controlled clinical trials that evaluated the effect of elevation or positioning of the head of the bed on relieving symptoms in adults. This review refers to certain methodological limitations and the available literature that prevented reliable conclusions from being drawn about the effect of elevating the head of the bed on the relief of gastroesophageal symptoms. However, head of bed elevation could be considered a cheap, relatively safe and promising alternative to pharmacological interventions with unfavorable long-term safety profiles. It can even be considered as an adjuvant in severe cases where surgery needs to be deferred due to the patient's characteristics, favoring a reduction in the dose of pharmacological treatment.

Postural orthostatic tachycardia syndrome (POTS) is a common complex disorder of the autonomic nervous system, also known as dysautonomia, estimated to affect 1 to 3 million people in the United States as of 2019. People of any age, sex or race can be affected. There is no cure for any form of dysautonomia at this time, but despite the high prevalence of dysautonomia, most patients take years to be diagnosed due to a lack of awareness among the public and the medical profession.

Dysautonomia is a condition of the autonomic nervous system (ANS). The ANS controls many automatic processes vital to the body, such as blood pressure, heart rate, breathing, body temperature, digestion, sweating, urination (urination), and sexual response. Dysautonomia can affect all or part of the ANS, and sometimes causes serious problems, such as heart and blood pressure disorders, difficulties breathing or swallowing, and impotence in men. There are many types of dysautonomies. They may be inherited or caused by injury, or by conditions such as diabetes, Parkinson's disease, an autoimmune disease, or alcoholism. Sometimes the cause is unknown. Also called neurovegetative dysfunction and autonomic nervous system disorder.

There are situations that can aggravate or trigger syncope events, among these are dehydration, altitude (mountaineering), pregnancy, intense pain or acute intense emotion, acute anemia, among others. Sometimes an event can be preceded by diaphoresis, nausea, dizziness and yawning, however, on other occasions syncope occurs without prodromes.

The above allows us to identify that in the state of the art there are non-invasive alternatives that seek to provide options for the treatment and/or mitigation of the negative effects and/or symptoms that some disorders or diseases that appear or worsen during the period of treatment could have a patient's sleep or when lying in a horizontal position. In the state of the art there are multiple technologies that aim to mechanically treat the symptoms in patients suffering from the said disorders, some of them include tilting bed systems and frames, therefore, the analysis presented below focuses on highlight the main technical characteristics of those identified documents, which have a certain relationship with the proposed invention, highlighting the most important differences with respect to it, at the same time emphasizing the advantages offered by the device and method that is sought to be protected.

In this sense, U.S. Pat. No. 5,566,412 describes a bed frame with the ability to be raised at different inclinations so that the user has an inclined bed that minimizes the symptoms of gastrointestinal diseases during the night. Similar to this publication, other disclosures have made variations to pursue the same goal of positioning a patient's body in a specific inclination to treat a patient's digestive symptoms.

Among said documents we identified those referring to a certain degree of inclination, such as document RU2305532, which refers to a bed that, through wooden blocks, allows its inclination to be raised up to 5°; document U.S. Pat. No. 6,898,812 referring to a device that allows inclination in a range of 0 to 300 but is focused on infants; the invention described in CN217611828U which comprises a cushion with a fixed inclination of between 15 to 20°; further, document CN213883916U refers to a bed that can reach an inclination of up to 90°; document CN203790198U refers to a pillow that provides an inclination of between 35 to 45°; while the device described in U.S. Pat. No. 4,471,767 achieves a fixed inclination of 30°; in document U.S. Pat. No. 5,127,422, mention is made of a technology that allows inclination ranges of 30 to 450 to be achieved; Finally, document U.S. Pat. No. 5,528,783A refers to a device that allows reaching inclination ranges from 0 to 90°. Some other documents refer to tilt as a novel feature, however, do not claim specific tilt ranges, including: KR20230111622, KR102026706, CN211244193U, KR102370933, KR20110078599, JP2016067429, WO201469713, CA2836451, CN212699430U. Although some of these technologies refer to the inclination ranges of the present invention, they lack other novel characteristics of the latter, such as the configuration of the device or the method that allows its operation through manual, predetermined and control emergency routines.

Document JP2019503263 relates to an adjustable bed structure and method of operation, which includes multiple adjustable sections, where each section can be adjusted independently. In this invention the adjustable bed frame is coupled to a processor configured to collect biological signals associated with multiple users. This technology is tilted in three sections to treat two conditions that are practically associated with each other: hoarseness and respiratory apnea, while the invention proposed in this application is tilted completely to treat multiple conditions related to the posture of the human body: respiratory apnea, gastroesophageal reflux, dysautonomia, in addition to improving circulation, metabolism, respiratory function especially in pregnant women, neurological and immunological improvement, as well as facilitating the ascent and descent of people with some physical difficulty or disability.

The invention described in document JP2019503263 compares the critical values or of some crisis episode with the average values of the user himself, however the present invention, in addition to taking these values as a reference, are compared with the normal values of an average patient with similar characteristics and through standardized questionnaires and the comparison of the results of other users to do two things: the first, anticipate and prevent crisis episodes and the second, propose a personalized therapy for each user. Other differences found in the aforementioned document are that it does not mention the relationship between the inclination of the bed with respect to the condition that is trying to treat or alleviate, nor does it mention that it requires a routine or a therapy proposal for the user, nor the use of an application or user interface. It should be noted that the proposed invention relates and differentiates the degrees of inclination and angles allowed with the various conditions, with the aim of adequately treating or alleviating their symptoms; In addition, it allows the identification and implementation of routines adapted to the needs of each patient.

Moreover, in the state of the art, some patent documents were identified that refer to inventions that allow tilting, but that in their configuration comprise various sections, among which the following stand out: document KR20230111622 describes an invention with a sectioned structure, the operation of which do not includes the use of predetermined routines aligned with the symptoms of the condition of interest, its main objective is to care for patients who live in a bed and provide certain massages; The invention described in KR102026706 refers to a device that comprises various sections that allow it to be tilted, but it differs from the present invention because it does not include certain essential components, such as the digital interface or the possibility that it can interact with a mobile application executed on a mobile device, neither includes user profiles or routines for use; there is monitoring of all vital signs but, beyond seeking maximum comfort for the patient in bed, it is not specified what it does with them or why it collects the information from the variables obtained. The apparatus of document KR20110078599 is divided into three sections, which are moved by mechanical transmission mechanisms of conical gears and other elements, it also mentions a bio signal with piezoelectric sensors regarding the detection of urine and for detecting snoring, but does not describe its interaction with a control unit or data processing; The document WO201469713 refers to a device sectioned longitudinally into three parts, in addition to detecting episodes of respiratory apnea using noise sensors, helping the patient to turn in a longitudinal direction to lie on the shoulder and better ventilate the respiratory tract. Therefore, its main difference with the proposed invention is that its purpose is to accommodate the patient on the shoulder by tilting at least a part of the structure, allowing variables such as temperature and weight to be sensed, while the apparatus and method of this invention seeks to adapt the inclination of the bed taking into account variables that allow mitigating the symptoms of various chronic conditions, in addition to contemplating the use of emergency routines.

Patent document KR20230111622 refers to a reclining bed apparatus and method comprising a monitor, an application, and a server; It foresees the use of a schedule for programming, the use of bio signals and communication with a caregiver, however, it does not refer to the method comprising the use of routines to facilitate the treatment of the symptoms of a specific condition. Regarding the device, the cited document focuses on the care of patients who live in abed and provides them with certain massages, in addition to being sectioned and allowing remote connection, while the configuration of the proposed invention is focused on allowing its inclination based on information related to the vital signs that are measured recurrently by the system, which allows the use of various routines that allow mitigating the symptoms of various chronic conditions.

Furthermore, patent document AR063733 refers to a tilting bed by gravity, which allows the user to tilt the entire support plane of the mattress, raising its headboard, by displacing his own weight in the opposite direction to that of said headboard. In addition, it allows the user to return to the horizontal position, without moving, by pressing a command located within reach. However, it does not have the novel characteristics and components that are intended to be protected with the invention proposed for registration, particularly, the configuration of the device that integrates the bed has a different configuration, with components and structures that differ from one invention to another, since the aforementioned invention is completely inclined by an axis and an oleo-pneumatic (oil-air) piston whose flow is controlled by a button panel. Moreover, it does not describe a method for bed control, nor the use of specific routines to alleviate the symptoms of certain conditions, so it does not affect its patentability, although it does constitute a precedent with similar characteristics.

In the other hand, document U.S. Ser. No. 00/677,2462B1 refers to a tilting bed, which describes a bed assembly that has a horizontal base frame and a second frame articulated at one end to the base frame and an inflatable bag adjacent to the other end for raising and lower one end of the tilting frame with respect to the base frame. A three-position manual switch is connected via a relay to an electrically operated three-position solenoid valve and an air compressor to selectively raise and lower one end of the bed at the will of the person lying in the bed. Alternatively, instead of an inflatable bag, an electric actuator may be provided to raise and lower one end of the tilting frame relative to the base frame. A conventional foundation is received on the second frame with a stop on the second frame to prevent it sliding off when the frame is tilted.

Regarding the differences with the proposed invention, in the referred document the control is 100% analogous, since it is controlled by relays, which limits its functionality and the possibility of being integrated with digital platforms. Therefore, it does not allow the integration of routines, nor does it facilitate having multiple users. Another important difference lies in the pivot point of both inventions, the one described in the cited document is located at one end of the structure, which creates a lever arm in any of the action mechanisms that it has (inflatable or linear movement), forcing them to be much larger, noisier, and less efficient in energy consumption. In the case of air bags, a compressor is required, which causes large amounts of noise that makes their daily use for rest difficult.

As a summary, in the state of the art multiple technologies are identified with technical characteristics similar to those of the proposed invention, those previously analyzed are the closest, but others are discussed below that support the technical contribution in the technology invention that is intended to be protected.

Document KR20160080898 refers to an invention that comprises a pair of pressure sensors in the bed, for measuring various indicators, both vital signs and the environment to control the position of at least one part of the bed, but it is not linked to the treatment of chronic diseases. Patent JP2016027897 describes a device that tilts in three sections to help the patient move the body to avoid pressure at a specific point, which is useful for some sleep disorders, so its operation is used to open the patient's airways but it does not include the use of routines for the treatment of specific conditions.

Document JP2012502671 describes a device that monitors heart and respiratory rate, as well as blood pressure, detects movement in bed with motion, pressure, and visual recognition sensors, and also has microphones to detect noise and monitor hypoglycemia and the tremor or the absence or presence of the patient. The device can generate sound and motion alerts, provide vibration and massage therapies, and can tilt the back of the bed. However, it does not refer to a method that allows the use of routines for the treatment of specific conditions, defined manually or automatically, therefore, it does not treat the conditions to which it refers, but rather avoids crisis episodes by sending emergency signals to alert the caregiver or the patient when they occur.

Patent IN202147020955 refers to a smart bed system that measures various vital signs, but there is no relationship between these variables and the user's inclination, which is carried out thanks to air balls that are inflated in strategic places under the mattress. The invention of document CN205126253U refers to a system to assist in episodes of apnea, which includes an emergency routine that consists of tilting the patient and notifying their caregivers, however, it does not include a device that allows the implementation of said routine, nor does it refer to other conditions. Document CN215020808U describes a technology that allows respiratory distress to be prevented by means of a lateral inclination mechanism of the mattress, which implies that the configuration of the device is different from that proposed in the present invention, in addition to the fact that it does not include the use of routines for the treatment of various chronic conditions.

Document JP2022527875 includes the measurement of various parameters of the environment in which the bed is located, such as air quality, light, sound, humidity, temperature, among others, but it does not include the monitoring of the user's vital signs, so it does not affect the novelty of the invention proposed for registration. The technology disclosed in CA2989625 refers to a method that helps improve the user's sleep quality by measuring some environmental variables and taking certain actions to improve these conditions, but the only action included is the inclination of the head of the bed, without that is related to environmental conditions, therefore, its use is not linked to the treatment of chronic conditions through specific routines.

Documents CN114432060, CN213851555U, CN214549919U, CN218187165U, CN114948480, CN211131790U, CN211187928U, CN218552629U, CN211131789U, U.S. Pat. No. 5,127,422, and CN113768716 refer to methods and/or devices focused on avoiding reflux episodes in infants, including the possibility of changing or moving position of the infants to avoid their potential deadly consequences. These technologies do not refer to other chronic conditions, so they do not include routines in their operation to facilitate the treatment of these, in addition to the fact that due to the physical characteristics of the users they are focused on, they require that the device have a particular configuration, which is different from that of the present invention.

Documents WO200164103 and U.S. Ser. No. 00/734,6951B1 refer to an invention that allows the monitoring of invalid patients, therefore it includes sensors for measuring various variables and sending information through a computer system to their caregivers; in the case of the first document, it does not allow a tilt; while the second does allow controlled inclination in the transverse direction and partially in the longitudinal direction, with the aim of relieving the support against the mattress to avoid the formation of sores. In both cases they do not include the use of routines related to certain conditions and the configuration of the device is different from that proposed in this invention.

The inventions of documents CN219332258U and CN218792834U refer to inflatable devices to facilitate blood circulation in the user's limbs, to avoid an episode of venous or vascular reflux, but their use is not linked to the chronic conditions to which they refer. The present invention does not include the structure or operation proposed herein.

On the other hand, inventions were identified that refer only to inflatable structures or mattresses with a certain configuration for the treatment of reflux, which are described in CN209808816U, CN203724377U, CN215081233U and CN204600970U. Although these documents describe technologies that help prevent the symptoms of this chronic condition, they do not include the measurement of vital signs, the analysis of these values for the patient's inclination through manual or pre-designed routines, so they do not constitute obstacles to the present invention.

As shown from the analysis above, in the state of the art there is still a need for other non-invasive ways to treat the symptoms of the previously mentioned conditions, which do not require an external device, invasive technology or pharmaceutical treatment, or that can complement the action of these measures. These alternatives must allow measurement, adaptation, and personalization of treatment without disturbing the patient's sleep. The deficiencies in the documents analyzed can be summarized in that they do not have a configuration similar to that of the present invention, but rather they propose the elevation and/or inclination of the bed through various mechanisms, which require that the structure of the bed is divided or integrated in different ways; Nor do they include the use of information collected from vital signs acquired manually or automatically to modify the elevation of the bed, according to the patient's needs; On the other hand, they do not contemplate the use of routines that facilitate attention to the symptoms of the disorders or diseases on which the present invention focuses.

The present invention constitutes a novel and non-obvious alternative that resolves the existing deficiencies in the state of the art, since existing technologies do not have the capacity to improve their relief effect depending on the symptomatic severity of the health conditions. of the patient, therefore, it is a non-invasive technology to prevent and alleviate the symptoms of chronic diseases and disorders such as dysautonomia, obstructive sleep apnea and gastroesophageal reflux disease (GERD), among others.

### OBJECT OF THE INVENTION

It is an object of the present invention to provide a tilting bed and a method for treating and alleviating the effects and risks of gastrointestinal, respiratory, and cardiovascular disorders, by the degree of inclination of the bed that results in an inclination of the body so that it is always is straight and without the need to bend as in the case of a hospital bed.

Another object of the present invention is to provide a closed-loop feedback system that adjusts treatment routines according to standardized questionnaires that allow for personalized adjustment of tilt therapies as well as the application of rescue and emergency routines by monitoring vital signs that can be collected with smart watches and/or through peripheral accessories involving a central processing unit that is in constant communication with a server hosted in the cloud.

The user has the possibility of sharing their use values with this system for the collection of information and Big-Data analysis where the values of all users are consolidated to help enrich the repository of routines, as well as prediction behavior through artificial intelligence algorithms, with the aim of proposing routines to users that are more appropriate to their needs and physical characteristics.

Other objects will be noted later in the description of the invention or will be apparent to those skilled in the art.

### BRIEF DESCRIPTION OF THE DRAWINGS

The aforementioned and other features of this disclosure and the manner of obtaining them will become more apparent, and the disclosure itself will be better understood with reference to the following figures, which constitute illustrative and non-limiting examples.

FIG. 1 is a perspective view of one embodiment of the tilting bed of the present invention, which comprises the closed-loop feedback system in a non-elevated initial position;

FIG. 2 is a perspective view of one embodiment of the tilting bed of the present invention, which comprises the closed loop feedback system in an elevated position;

FIG. 3 is a top view of one embodiment of the structures that support the mattress of the tilting bed of the present invention;

FIG. 4 corresponds to a block diagram illustrating the method of operation of tilt therapy in combination with vital signs monitoring.

FIG. 5 shows an example routine of a seven-hour period for a respiratory apnea disorder that includes a standard routine through the method for treating symptoms of patients with chronic disorders of the present invention, as long as the individual's oxygenation levels do not decline of the minimum standard in accordance with the user's own parameters and configuration, the routine will continue according to the previously selected program.

FIG. 6 illustrates an example of a routine for a period of a few minutes for a respiratory apnea disorder that includes a standard routine implemented through the method for treating symptoms of patients with chronic disorders of the present invention, it can be seen that the the individual's oxygenation values dropped abruptly, possibly due to an episode of apnea, and at this moment an emergency routine is activated which takes precedence over any other routine to try to restore normal oxygenation values in a phased manner. Once normal values are recovered, the dynamic routine or the one selected by the user will continue.

FIG. 7 illustrates an example of a routine of a period of a few seconds for a respiratory apnea disorder that includes a standard routine implemented through the method for treating symptoms of patients with chronic disorders of the present invention, this figure shows the most critical scenario where the activation of an emergency routine was not enough to recover the oxygenation levels in the individual, it is when the emergency protocol is activated, which consists of activating abrupt movements to try to wake the individual so that they do not collapse in a prolonged episode of respiratory apnea with a catastrophic end for the user.

The examples set forth herein illustrate embodiments of the invention that should not be construed as limiting the scope of the disclosure in any way. Additional features of the present disclosure will become apparent to those skilled in the art upon consideration of the following detailed description of illustrative embodiments that exemplify the best mode of carrying out the disclosure as currently perceived.

### BRIEF DESCRIPTION OF THE INVENTION

The present invention relates to a tilting bed for treating and alleviating the effects and risks of gastrointestinal, respiratory, and cardiovascular disorders, through elevations of the bed that result in an inclination of the body, which includes:
A mattress (126) for the rest of a patient with chronic disorders and diseases;
A support structure for the mattress (122) that allows the mattress (126) to be secured and maintained in position, in addition to facilitating its tilting;
Perpendicular support elements (116) for supporting and elevating the mattress support structure (122);
Rotating elements (118) to facilitate the rotation of different components of the invention;
A circular bar (120) that allows the rotation of the mattress (126) in one degree of freedom in the longitudinal direction of the mattress;
A horizontal support structure to the floor (124) that provides support and connection to the invention;
A foot structure (102) that provides stability and prevents the invention from overturning;
A lifting element (104) comprising five control elements for moving up or down the position of the mattress (126) and the support structure for the mattress (122), including an actuator (106) for raising the structure of support for the mattress (122) to a certain position; a computer or information processing and control unit (110), which is responsible for controlling the position of the linear actuator (106) and supplying the energy it requires for its operation; a power supply, which supplies energy to the computer; a device for measuring vital signs (111), such as oxygenation, heart rate, respiratory rate and temperature, which can be an accessory or external device or peripheral device like the one of the present invention; a server connected to the internet (112);
A mattress retaining element (128) to prevent sliding of the mattress (126);
A module for wired control of the bed through a digital interface (114).

The invention also relates to a method for treating and alleviating the effects and risks of gastrointestinal, respiratory, and cardiovascular disorders, by means of bed elevations that result in a tilt of the body, which includes the following steps:
a) creation of a user profile through a digital interface (210);
b) selection of the routine that will be implemented by the device of the present invention, which takes into consideration two variables that are: inclination and time; This stage includes the identification and selection of a predefined routine, the manual entry of the necessary parameters to define a routine according to the user's needs (214), or the continuation of a dynamic routine (218-220-222-224-246), the dynamic routine is that routine that changes according to the adjustments made through the questionnaires, the comparison of the parameters and the particularities of each user;
c) confirmation and start of the selected routine (216);
d) measurement of the user's vital signs data provided by a smart watch or through the peripheral device for measuring vital signs (111), comparison with the user profile data initially recorded and sending a signal of the measured vital signs to the digital interface to identify if there is a need to adjust the elevation of the bed (244);
e) lifting the bed structure according to the parameters of the defined routine (220);
f) end of routine (222);
g) entry of information regarding the status of the patient's symptoms (224);
h) routine adjustment based on user feedback.

### DESCRIPTION OF THE INVENTION

While the present disclosure may be susceptible to embodiments in different forms, the figures show, and herein describe in detail, embodiments with the understanding that the present descriptions should be considered exemplifications of the principles of the disclosure and are not intended to be exhaustive or limit the disclosure to the construction details and component arrangements set forth in the following description or shown in the figures.

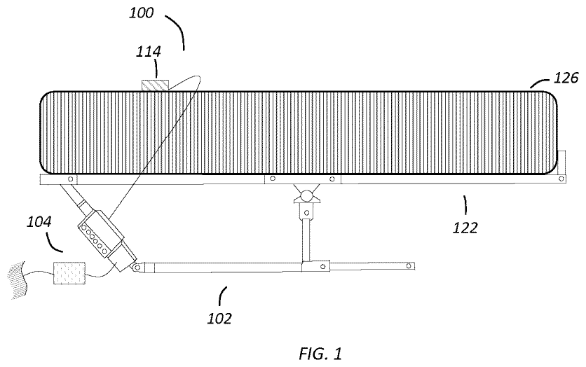

As can be seen in FIG. 1, the invention begins from a flat sleeping position perpendicular to the floor, and comprises a mattress (118) for the rest of a patient with chronic disorders and diseases; a support structure for the mattress (122) that allows the mattress (126) to be secured and maintained in position, in addition to facilitating its tilting; perpendicular support elements (116) for supporting and elevating the mattress support structure (122); rotating elements (118) to facilitate the rotation of different components of the invention; a circular bar (120) that allows the mattress (126) to rotate in one degree of freedom in the longitudinal direction of the mattress; a horizontal support structure to the floor (124) that provides support and connection to the invention; a foot structure (102) that provides stability and prevents the invention from overturning; a lifting element (104) comprising control elements for moving up or down the position of the mattress (126) and the support structure for the mattress (122), including an actuator (106) for raising the support structure for the mattress (122) to a certain position; a mattress retaining element (128) to prevent sliding of the mattress (126); and a module for wired control of the bed through a digital interface.

The support structure for the mattress (122) comprises rectangular square profiles, which are arranged so that they form a structure that supports the mattress (126), so they can adapt to the shape and size of said element. Inside said support structure (122) and attached to its frame, the horizontal support structure for the floor (124) is arranged, which preferably has the shape of an asterisk. The profiles that comprise both structures are assembled telescopically through mechanical fastening means and are held in position by a safety pin that blocks movement.

The support structure for the mattress (122) and the horizontal support structure for the floor (124) have attached, through mechanical fastening means, a circular bar (120) to the lower part that is supported by a pair of rotating elements (118) that allow rotation in one degree of freedom in the longitudinal direction of the mattress. The support structure for the mattress (122) is attached to the upper part of the lifting element (104), which allows it to rotate up to 21° from its horizontal position (0°). Likewise, the support for the mattress (122) includes on the upper face of its lower end the mattress retaining element (128) to prevent the displacement of the mattress (126) when the support structure (122) begins to tilt.

The pair of support elements perpendicular to the floor (116) have the function of supporting and elevating the support structure (122) with the pair of rotating elements (118). The perpendicular support elements (116) consist of a structure of rectangular square profiles that are assembled telescopically and held in position by a safety pin that blocks movement. The perpendicular support elements (116) are joined through mechanical fastening means in their lower part to the foot structure (102) that provides stability and support to the entire invention, while in their upper part to the pair of rotating elements (118).

Preferably each of the pair of rotating elements (118) is composed of a pair of pedestal bearings. In their lower section, these bearings are screwed and attached to a base capable of being telescopically assembled to the pair of perpendicular support elements (116) and ensuring their movement using a safety pin. The pair of rotating elements (118) are aligned by their rotating element (118) to receive the circular bar (120) attached to the bottom of the support structure (122).

The foot structure (102) functions as support and anchoring of the entire structure. On the one hand, it is attached to the perpendicular support elements (116) and on the other it is attached to the box, skirting board, bed furniture or to the floor directly. Preferably, the foot structure (102) has four extensions or telescopic feet that provide stability and prevent the invention from tipping over.

The lifting element (104) in turn consists of four elements (106, 108, 110 and 111) connected, preferably by cables, and a fifth wireless and remote element which is the server (112). The first of said elements is a linear actuator (106) that has the function of supporting and tilting the bed in a range of 0 to 21° with respect to the horizontal position. This element is attached at the top to the mattress support structure (122) by means of a bolt that allows the rotational interaction of these two elements. At the bottom it is attached to the foot structure (102) by means of a bolt that allows the rotary interaction of these two elements.

The second element (110) is a computer for data processing and control unit, which is responsible for controlling the position of the linear actuator (106) and supplying the energy it requires for its operation. The actuator (106) is wired to said computer or processing unit. Likewise, the computer or control unit is connected by wire to the remote control module (114) to receive direct instructions from the user and provide immediate response to activate the movement of the actuator (106).

Preferably, the computer or control and processing unit has WiFi and Bluetooth communication protocols, capable of communicating with a mobile device and/or directly with a server dedicated to the processing and storage of information that the user can consult, program. and interact with the device from anywhere in the world.

The third element (108) is a power supply, which supplies energy to the computer. The fourth element is a device for measuring vital signs (111), which may be an accessory or external device or peripheral device of the present invention. The fifth element (112) is a server connected to the internet.

The control module (114) is a wired control that allows the movement of the actuator to be controlled at any time. This module connects directly to the computer or information processing and control unit (110).

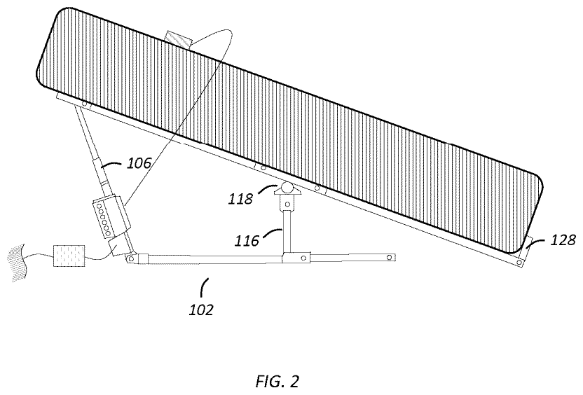

FIG. 2 shows an example of a preferred embodiment of the bed of the present invention in an elevated position, in which the angle of the mattress structure (126) may be greater than zero but less than twenty-one degrees, depending on the input of the user. The vertical structure (102) includes the pair of support elements perpendicular to the floor (116) that include a rotating element (118) that allows the support for the mattress (122) to rotate in its longitudinal direction between a flat position at an angle of zero degrees and an inclined position of maximum 21°.

The angle of the mattress support structure (122) can be modified in several ways. The first of them, through the remote control (114); The second way to modify the inclination of the structure is to agree to start a predefined routine or designed directly by the user, or the continuation of a dynamic routine. The mattress support (122) can also move automatically when-through a peripheral device (111) or a smart watch—an anomaly is detected that warrants the activation of a rescue routine or an emergency routine. A final way to control the movement of the structure will be through voice commands through a smart home management device, such as Alexa.

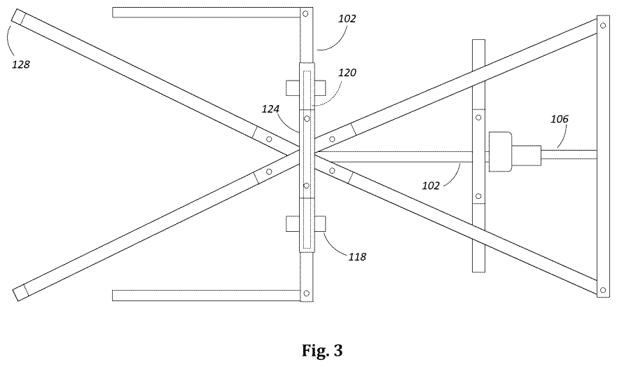

FIG. 3 shows a preferred embodiment of the structures that support the mattress (126) of the bed of the present invention at an angle of inclination allowed by the present invention, particularly the support structure for the mattress (122), the rotating elements (118), the circular bar (120), the horizontal support structure to the floor (124), the standing structure (102) and the lifting element (104).

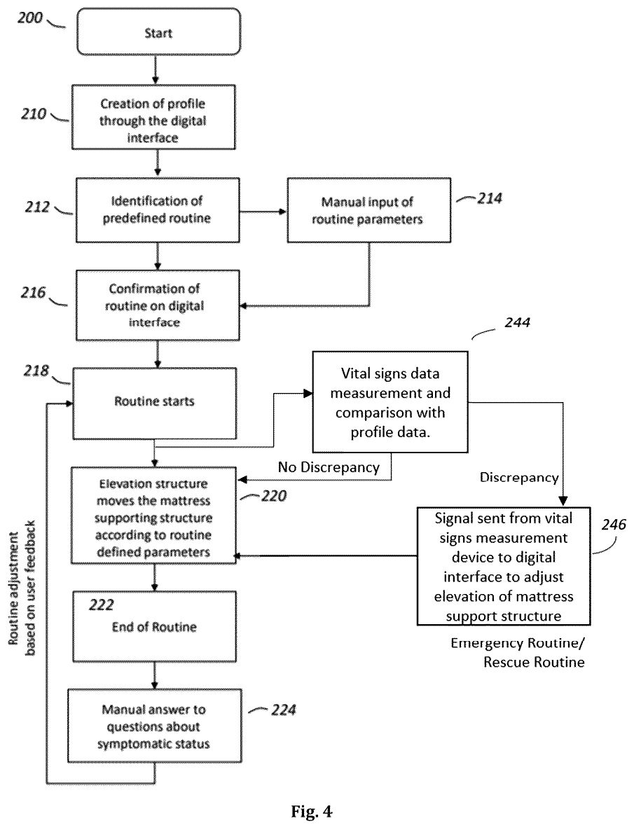

FIG. 4 comprise a block diagram of the method for treating and alleviating the effects and risks of gastrointestinal, respiratory, and cardiovascular disorders, by means of bed elevations that result in an inclination ranging from 0° to 21° with respect to the horizontal, which includes the following stages:
a) creation of a user profile through a digital interface (210);
b) selection of the routine that will be implemented by the device of the present invention (212), which takes into consideration two variables that are: inclination and time; This stage includes the identification and selection of a predefined routine, the manual entry of the necessary parameters to define a routine according to the user's needs (214), or the continuation of a dynamic routine (218-220-222-224-246), the dynamic routine is that routine that changes according to the adjustments made through the questionnaires, the comparison of the parameters and the particularities of each user;
c) confirmation and start of the selected routine (216);
d) measuring the user's vital signs data, comparing it with the user profile data initially recorded and sending a signal of the measured vital signs to the digital interface to identify if there is a need to adjust the elevation of the bed (244);
e) lifting the bed structure according to the parameters of the defined routine (220);
f) end of routine (222);
g) entry of information regarding the status of the patient's symptoms (224);
h) routine adjustment based on user feedback.

The step 210 of creating a user profile is carried out by the server (112) and the computer or information processing unit (110), or through a mobile application executed on a mobile device, which communicates with said computer (110). To carry out this stage, a digital interface is required to which the user accesses and provides personal data of the patient or the user of the invention. Preferably, the data provided includes general information about medical condition, symptoms, treatment, among others; Likewise, it generates a username and password. All data collected is stored on the server (112) to facilitate subsequent consultation.

At this stage, the user decides whether to grant permissions so that other mobile devices can access the information stored in their profile, such as mobile devices, watches or smart bracelets, which allows them to share the information they collect through their APIs with the computer (110) and the server (112) of the invention, to enrich and provide feedback on its operation, as well as for the creation of comparative tables.

Step 212 comprises the selection of the routine that will be implemented by the device of the present invention, which comprises three alternatives: a) the selection of a predefined routine, b) the manual definition of a routine according to the user's needs, or c) continuation of a dynamic routine.

The first option relating to the routine of a plurality of routines that are predefined. These routines are created from the symptoms of the different disorders and diseases for which the invention is directed. They are designed based on the results of previous scientific research on the subject and are chosen from those existing in the state of the art. For example, these predefined routines include the following disorders or chronic conditions: GERD, POT, OSA, as well as some respiratory problems such as hoarseness, nasal congestion, among others. It also helps exercise the heart and accelerate metabolism during rest.

The second option includes providing the user with the option to define their own routines according to their needs and those of the condition they wish to treat. To do this, the digital interface executed on the computer (110) or in the mobile device application.

The third option includes the selection by the user, through the digital interface, of a dynamic routine (218-220-222-224-246), which consists of a previously used routine that has been fed back and adapted to their needs and habits, which requires the application of periodic questionnaires, as well as artificial intelligence algorithms, which analyze and contrast the user's data, the data on their vital signs collected periodically thanks to the use of devices that monitor said signs of the patient on a recurring basis, as well as scientific data related to the symptoms and treatments of the various conditions on which the invention focuses.

Step 216 comprises confirming the predefined or user-defined routine through use of the digital interface.

Step 218 comprises the start of a routine. When the user is ready to start their rest hours on the device, they can use the application, the web version or even through a voice command (if they have a smart home administrator, e.g. Alexa) to start to routine.

Step 220 comprises raising the bed according to the parameters established by the executed routine, whether manually defined, predefined or dynamic, that is, comprising the feedback of vital signs measured periodically. The computer or processing unit (110) sends, through the server (112), the instructions to the linear actuator (106), so that it extends or retracts, which will cause its angle to change, which It is in constant communication with the server, which will result in alleviating the symptoms or discomforts that the user may be going through during their sleeping hours, improving their rest and general health performance.

Once the user's rest time has elapsed, stage 222 of the method of the present invention begins, which includes the completion of the routine executed on the device, the processing unit (110) sends the instruction to the actuator (106) so that return to initial horizontal position; or, automatically, for which it is required that the implemented routine comprise a period of time, preferably the duration of the routine is 8.5 hours, but it can be extended or reduced.

Step 224 includes the entry of information regarding the status of the patient's symptoms, which begins once the routine is completed. The user, through the digital interface on the computer (110) or the application on the mobile device, has the option of providing their opinion regarding the performance of the routine and their symptoms, which will be stored on the server (112) and processed by the computer (122), updating the user profile information for analysis, comparison so that it is desired, the following times select to continue with a dynamic routine, increasingly adjusted to the needs of the user.

Finally, the loop closes with adjustment of the routine based on user feedback. With the above information, predictions are made that impact the operation and performance of the device of the present invention, in the following routines to be implemented. Likewise, routines that are more beneficial are proposed to the user.

Emergency Routine and Rescue Routine

Step 244 comprises measuring vital sign data of the user. If the user has a smart watch and access to it was guaranteed during registration, or they have their own peripheral accessory or peripheral device for measuring vital signs (111). The information collected by the aforementioned devices is compared with the previously entered data (214), with the normal parameters of a person with the same characteristics, through the algorithms and programs of the processing unit (110) and/or the server. (112).

Step 246 comprises sending a signal of the vital signs measured through some device to the digital interface to adjust the elevation of the bed; The information collected above is compared with the normal values of a person with the same characteristics and through a comparative algorithm the decision is made to continue with the predefined routine or activate an emergency routine or even the rescue routine in the presence of an episode of crisis.

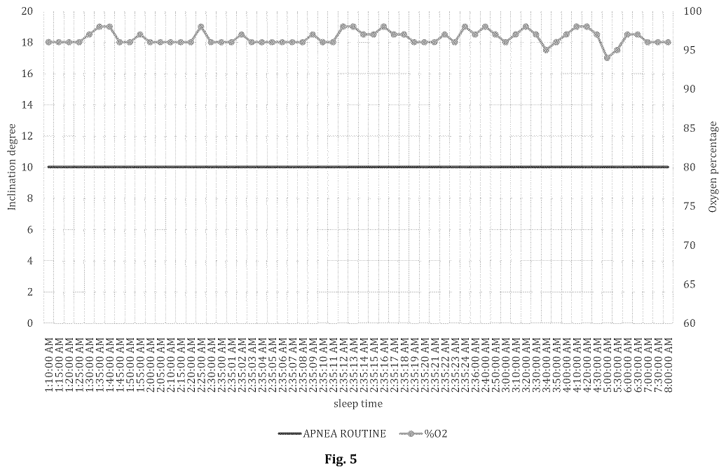

FIG. 5 illustrates an example routine for a seven-hour period for a respiratory apnea disorder that includes a standard routine, for which there is a device capable of measuring the individual's oxygenation in real time. If the individual's oxygenation levels do not fall below the minimum standard according to the user's own parameters and configuration, the routine will continue according to the previously selected program.

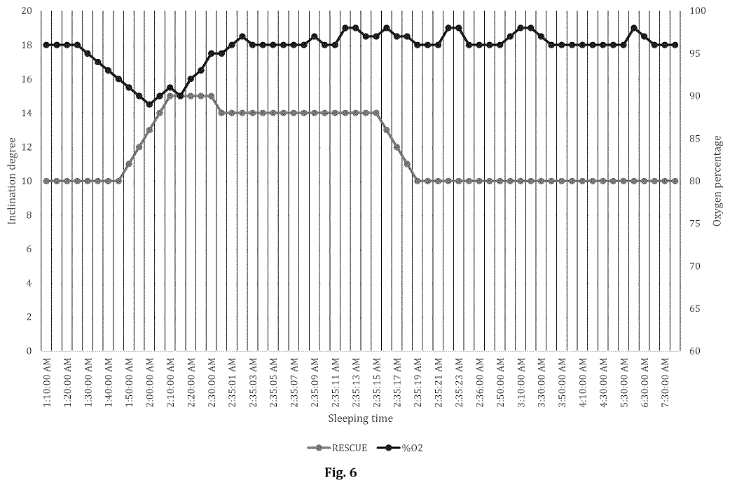

FIG. 6 illustrates an example of a routine for a period of a few minutes for a respiratory apnea disorder that includes a standard routine and also has a device capable of measuring the individual's oxygenation in real time. For this second scenario, the individual's oxygenation values dropped abruptly, possibly due to an episode of apnea, and at this moment an emergency routine is activated which prevails over any other routine to try to restore normal oxygenation values in a phased manner. Once normal values are recovered, the dynamic routine or the one selected by the user will continue.

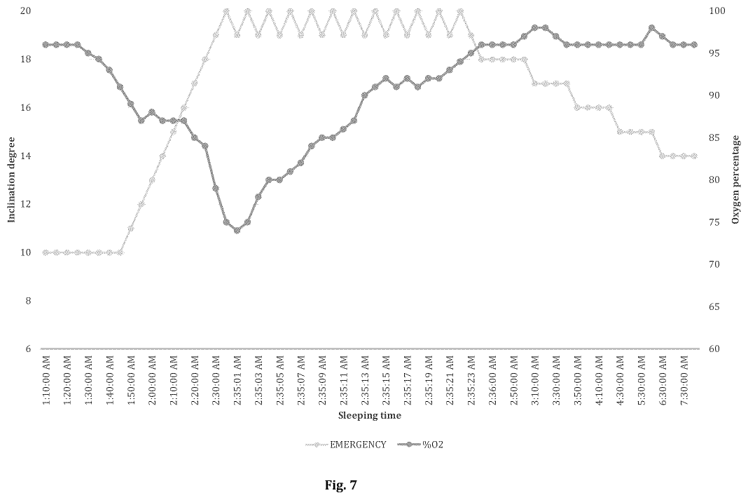

FIG. 7 illustrates an example of a routine of a period of seconds difference for a respiratory apnea disorder that includes a standard routine and also has a device capable of measuring the individual's oxygenation in real time. This is the most critical scenario where the activation of an emergency routine was not enough to recover the oxygenation levels in the individual. This is when the emergency protocol is activated, which consists of activating abrupt movements to try to wake the individual so that they do not decay into a prolonged episode of respiratory apnea with a catastrophic end for the user.

Some modalities of the present invention have been described, which are intended to cover any variation, use or adaptation using its general principles. It is anticipated that those skilled in this technical field may devise various modifications and equivalents without departing from the spirit and scope of the disclosure as enumerated in the following claims. This invention is intended to cover those variations of the present disclosure that fall within known or customary practice within the art to which it belongs.

1. An apparatus, namely a tilting bed for patients with chronic disorders characterized by:
a mattress (126);
a support structure for the mattress (122), which in turn comprises rectangular square profiles, assembled telescopically through mechanical fastening means and held in position by a safety pin that blocks movement; said profiles are arranged so that they form a structure that supports the mattress (126)
a horizontal support structure to the floor (124), which is fixed to the inner part of the frame of the support structure for the mattress (122), which is composed of rectangular square profiles, assembled telescopically through means mechanical clamps and are held in position by a safety pin that blocks movement
a circular bar (120)
rotating elements (118) which include pedestal bearings, in their lower part they are screwed and attached to a base and are aligned to hold the circular bar (120);
a lifting element (104) which in turn comprises four elements (106, 108, 110 and 111) connected to each other in a wired manner and a fifth wireless and remote element (112);
where the first element is a linear actuator (106) that has the function of supporting and tilting the bed in a range of 0 to 21° with respect to the horizontal position, and is attached to the upper part to the support structure of the mattress (122) by means of a bolt that allows the rotary interaction of these elements, while in its lower part it is attached to the perpendicular element (116) by means of a bolt that allows the rotary interaction of these two elements;
where the second element (110) is a computer or control and information processing unit to control the position of the linear actuator (106) and supply the energy it requires for its operation; Likewise, it is wired to connect to the control module (114) to receive direct instructions from the user and provide immediate response to activate the movement of the actuator (106);
where the third element (108) is a power source that supplies energy to the computer control and processing unit;
where the fourth element (111) is a peripheral device for measuring vital signs, such as oxygenation, respiratory rate, heart rate, temperature, etc.
where the fifth element (112) is an internet server.
a mattress retaining element (128) to prevent sliding of the mattress (126);
a foot structure (102) comprises four extensions or telescopic feet that provide stability and prevent overturning;
perpendicular support elements (116), which comprise a structure of rectangular square profiles that are assembled together telescopically and held in position by a safety pin that blocks movement; said support elements (116) are attached through mechanical fastening means, in their lower part, to the foot structure (102), while in their upper part to the pair of rotating elements (118);
a module for wired control of the bed (114) through a digital interface, which includes a pair of buttons that allow controlling the movement of the actuator at any time;
wherein the support structure for the mattress (122) and the horizontal support structure for the floor (124) have attached to their lower part, through mechanical fastening means, the circular bar (120) that is supported by a pair of rotating elements (118) that allow rotation in a degree of freedom in the longitudinal direction of the mattress;
where the support structure for the mattress (122) is attached to the upper part of the actuator (106), which allows it to make turns of up to 21° from its horizontal position (0°), as well as including on its face upper of its lower end the mattress retaining element (128) to prevent the displacement of the mattress (126) when the support structure (122) begins to tilt;
where the pair of support elements perpendicular to the floor (116) have the function of supporting and elevating the support structure (122) with the pair of rotating elements (118);
where the computer or control and processing unit has WiFi and Bluetooth communication protocols, capable of communicating with a mobile device and/or directly with a server dedicated to the processing and storage of user information.
where the control module (114) connects directly to the computer or control and information processing unit (110) of the lifting element (104).

2. A method to treat symptoms of patients with chronic disorders characterized by the following stages:
creation of a user profile through a digital interface, which is carried out through the server (112) and the computer or information processing and control unit (110), or through a mobile application executed on a mobile device, which communicates with said computer (110); where to carry out this stage a digital interface is required to which the user accesses and provides personal data of the patient or the user of the invention, the data provided includes general information about their medical condition, their symptoms, their treatment, among others; Likewise, it generates a username and password, the data collected is stored on the server (112) to facilitate subsequent consultation;
selection of the routine that will be implemented by the device of the present invention, which takes into consideration two variables that are: inclination and time; This stage includes the identification and selection of a predefined routine, the manual entry of the necessary parameters to define a routine according to the user's needs, or the continuation of a dynamic routine;
wherein the option relating to the predefined routine comprises the selection of a routine from a plurality of routines, through the digital interface, which are predefined and preloaded in the computer or control and information processing unit (110) or the mobile device application;
where the second option includes providing the alternative to the user to define their own routines according to their needs and those of the condition to be treated, for which the digital interface executed on the computer or control and information processing unit (110) or in the mobile device application, it collects a series of additional data related to various variables required to define the operation of the device, such as time, inclination, elevation, among others;
where the third option includes the selection by the user, through the digital interface, of a dynamic routine, which consists of a previously used routine that has been fed back and adapted to their needs and habits, for which it is required from the application of periodic questionnaires, as well as artificial intelligence algorithms stored in said devices, which analyze and compare the user's data, the data on their vital signs collected periodically thanks to the use of devices that monitor said signs of the patient on a recurring basis, as well as scientific data related to the symptoms and treatments of the various conditions on which the invention focuses;
confirmation and start of the selected routine, which includes the confirmation of the predefined or user-defined routine through the use of the digital interface of the computer or the information processing and control unit (110) or the mobile device and the start of this by sending the instructions required to comply with the variables and parameters included in the selected routine to the linear actuator (106); Once the user has routines stored in the memory of the computer or control and processing unit (110), they can execute them through voice commands;
measuring the user's vital signs data, comparing it with the initially recorded user profile data and sending a signal of the measured vital signs to the digital interface to identify if there is a need to adjust the bed elevation
where peripheral devices (111) or mobile devices are used to measure the user's vital signs, which are linked to the device of the present invention and the user's profile; The data collected by these devices are measured periodically and are sent, wired or wireless, to the server (121) or to the mobile device, to be viewed through the digital interface;
where the vital signs information collected by said devices is compared with the data previously entered, with those stored by the user or with the normal parameters of a person with the same characteristics, to act in any of the following alternatives: keep the routine running, adjust the elevation or tilt degree of the device or activate any predefined emergency or rescue routine;
elevation of the bed structure according to the parameters of the defined routine, which the computer or control and processing unit (122) sends, through the server (121), the instructions to the linear actuator (124), for it to extend or retract, which will cause its angle to change, which is in constant communication with the server (121);
end of the routine executed on the device, either manually, in which the mobile device, the computer or the control and processing unit (122) send the instruction through the server (121) to the actuator (124) so that it returns to its initial horizontal position; or, this stage can be executed automatically, for which it is required that the implemented routine comprise a period of time;
entry of information regarding the state of the patient's symptoms, in which the user, through the digital interface on the computer (122) or the application on the mobile device, provides feedback regarding the performance of the routine and its symptoms, which will be stored and processed by the memory of the computer or control and processing unit (122) and the server (121), updating the user profile information for analysis, comparison and processing when a new routine is executed;
routine adjustment based on user feedback;
where for the automatic adjustment of the routine, which includes the analysis, comparison and decision making related to the adjustment in the elevation and/or inclination of the devices, use of artificial intelligence is made.
